(19) United States
(12) Patent Application Publication
(10) Pub. No.: US 2022/0095916 A1
(43) Pub. Date: Mar. 31, 2022

(54) OPHTHALMOLOGIC APPARATUS

(71) Applicant: TOPCON CORPORATION, Tokyo (JP)

(72) Inventors: Yasuhisa ISHIKURA, Tokyo (JP); Wataru UMEJI, Tokyo (JP)

(21) Appl. No.: 17/481,612

(22) Filed: Sep. 22, 2021

(30) Foreign Application Priority Data
Sep. 28, 2020 (JP) 2020-161896

(51) Int. Cl. A61B 3/16

(52) U.S. Cl. CPC A61B 3/165

(57) ABSTRACT

An ophthalmologic apparatus that measures a value of intraocular pressure of an examinee's eye, based on a state of deformation of a cornea in applanation due to air blowing includes an air blowing unit that puffs air to the cornea and a control unit that controls an operation of the air blowing unit, wherein the control unit has a preliminary puffing mode in which preliminary puffing is executed such that the air blowing unit puffs the air in non-measurement of the value of intraocular pressure.

### CROSS-REFERENCE TO RELATED APPLICATION

The present application is based on and claims priority from Japanese patent application No. 2020-161896 filed on Sep. 28, 2020. the disclosure of which is hereby incorporated by reference in its entirety.

### FIELD OF THE INVENTION

A present disclosure relates to an ophthalmologic apparatus that measures a value of an intraocular pressure of an examinee's eye in a noncontact manner.

### BACKGROUND

An ophthalmologic apparatus that blows air to a cornea of an examinee's eye, and measures a value of an intraocular pressure of the examinee's eye in a noncontact manner based on a signal acquired by receiving reflection light of indicator light projected onto the cornea of the examinee's eye by is conventionally known (refer to JP5179894B, for example).

In the ophthalmologic apparatus that measures the value of the intraocular pressure by blowing air to the examinee's eye, secretions such as tears may scatter by the blew air, and the scattered secretions may blow into an air blowing unit via a nozzle. For this reason, the inside of the air blowing unit may be stained by the secretions.

The present disclosure has been made in view of the above circumference, and an object of the present disclosure is to provide an ophthalmologic apparatus capable of achieving inner-strain reduction of an air blowing unit.

### SUMMARY

To achieve the above object, an ophthalmologic apparatus according to the present disclosure provides an ophthalmologic apparatus that measures a value of intraocular pressure of an examinee's eye, based on a state of deformation of a cornea in applanation due to air blowing, the ophthalmologic apparatus includes an air blowing unit that puffs air to the cornea and a control unit that controls an operation of the air blowing unit, wherein the control unit has a preliminary puffing mode in which preliminary puffing is executed such that the air blowing unit puffs the air in non-measurement of the value of intraocular pressure.

### BRIEF DESCRIPTION OF DRAWINGS

FIG. 1 is a view schematically illustrating a configuration of an ophthalmologic apparatus a first embodiment.

FIG. 2 is a view illustrating an optical configuration of an intraocular-pressure measurement unit of the ophthalmologic apparatus of a first embodiment.

FIG. 3 is a view illustrating an air puffing configuration of the intraocular-pressure measurement unit of the ophthalmologic apparatus of the first embodiment.

FIG. 4 is a view illustrating an example of an operation button to be displayed on a display unit of the ophthalmologic apparatus of the first embodiment.

FIG. 5 is a view illustrating one example of a notice window to be displayed on the display portion of the ophthalmologic apparatus of the first embodiment.

FIG. 6 is a flowchart illustrating a flow of a preliminary-puffing-mode execution processing that is executed by the ophthalmologic apparatus of the first embodiment.

### DETAILED DESCRIPTION

With respect to the use of plural and/or singular terms herein, those having skill in the art can translate from the plural to the singular and/or from the singular to the plural as is appropriate to the context and/or application, The various singular/plural permutations may be expressly set forth herein for sake of clarity.

Hereinafter, an embodiment of an ophthalmologic apparatus of the present disclosure will be described based on a first embodiment illustrated in the accompanying drawings.

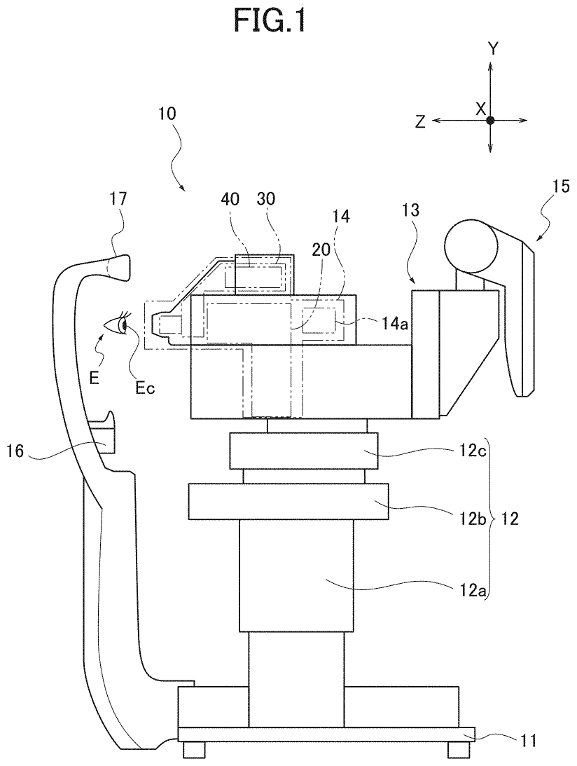

As illustrated in FIG. 1, an ophthalmologic apparatus 10 according to the first embodiment includes an intraocular-pressure measurement unit 14 that measures the value of intraocular pressure of an examinee's eye E in a noncontact manner. The ophthalmologic apparatus 10 includes a base 11, a drive unit 12 provided on the base 11, and an apparatus main body 13 supported by the drive unit 12. The intraocular-pressure measurement unit 14 is provided inside the apparatus main body 13. A display unit 15, a chin receiver 16, and a forehead support 17 are provided outside the apparatus main body 13.

The drive unit 12 includes a Y-axis drive part 12a, a Z-axis drive part 12b, and an X-axis drive part 12c, The Y-axis drive part 12a moves the apparatus main body 13 in the up-and-down direction (Y-axis direction). The Z-axis drive part 12b moves the apparatus main body 13 in the front-and-back direction (Z-axis direction (left-and-right direction in front view of FIG. 1)). The X-axis drive part 12c moves the apparatus main body 13 in the left-and-right direction (X-axis direction (direction orthogonal to a plane parallel to FIG. 1)). That is, the apparatus main body 13 is movable by the drive unit 12 in the up-and-down direction, the front-and-back direction, and the left-and-right direction orthogonal to the directions, with respect to the base 11.

The display unit 15 includes a liquid crystal display or the like and displays, for example, an image of the anterior segment of the examinee's eye E or a measurement result of the value of intraocular pressure of the examinee's eye E, The display unit 15 is supported turnable by the apparatus main body 13. For example, the display thereof can be oriented to the examinee, namely, the orientation of the display can be changed. The display unit 15 functions as a touch panel and thus serves as an operation unit that receives an input, such as an operation for measurement with the intraocular-pressure measurement unit 14 or an operation of manually moving the apparatus main body 13. The operation unit receives an operation input and outputs a signal of the received operation input to, for example, a control unit 34 to be described below. Note that, in a case where the apparatus main body 13 is equipped with, for example, a power button, an operation button, a measurement switch, and a control lever, the power button and the others are included in the operation unit.

The chin receiver 16 and the forehead support 17 are fixed to the base 11 and fix the position of the face of the examinee to the apparatus main body 13, for example, in measurement of the value of intraocular pressure. With the examinee having the face fixed by the chin receiver 16 and the forehead support 17, drive of the drive unit 12 causes the apparatus main body 13 to move with respect to the examinee's eye E. Note that the chin receiver 16 serves as a place on which the examinee rests its chin and the forehead support 17 serves as a place to which the examinee puts its forehead.

The ophthalmologic apparatus 10 according to the first embodiment has the display unit 15 on one side and the chin receiver 16 and the forehead support 17 on the other side across the apparatus main body 13. In normal use, the display unit 15 is on the side on which the examiner is located and the chin receiver 16 and the forehead support 17 are on the side on which the examinee is located. The apparatus main body 13 moves with respect to the base 11 due to the drive unit 12, proving with respect to the examinee's eye E (examinee's face) fixed by the chin receiver 16 and the forehead support 17.

Furthermore, although omitted in FIG. 1, the ophthalmologic apparatus 10 has a part ranging from the base 11 to the apparatus main body 13 over the drive unit 12, covered with a covering member in the external shape of the apparatus entirely. FIG. 1 illustrates the drive unit 12 having the Y-axis drive part 12a, the Z-axis drive part 12b, and the X-axis drive part 12c layered in the Y-axis direction. Here, the drive parts (Y-axis drive part 12a, Z-axis drive part 12b, and X-axis drive part 12c) are not completely divided in the Y-axis direction, and thus the parts are piled on one another when viewed in any direction orthogonal to the Y-axis direction.

The intraocular-pressure measurement unit 14 blows air to the cornea Ec of the examinee's eye E to bring the cornea Ec into applanation, and then measures the value of intraocular pressure of the examinee's eve E on the basis of the state of deformation of the cornea Ec in applanation. The intraocular-pressure measurement unit 14 includes a detection unit 20 and an air blowing unit 30.

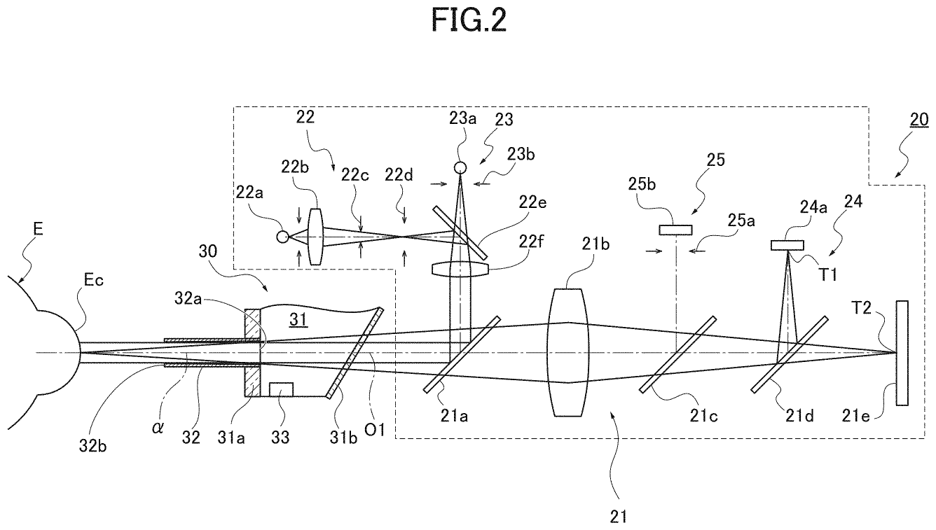

As illustrated in FIG. 2, the detection unit 20 includes an anterior-segment observation system 21, an XY alignment target projection system 22, a fixation target projection system 23, an XY alignment detection system 24, and a cornea deformation detection system 25. Note that the detection unit 20 may include a slit projection system that projects a pencil of slit-light on the cornea Ec, an optical reception system that receives light reflected off the surface or back face of the cornea Ec, or a Z alignment target projection system that aligns the detection unit 20 in the Z direction. Various types of detection results acquired by the detection unit 20 are input into a computing unit 14a (refer to FIG. 1).

The anterior-segment observation system 21 includes a first semi-transparent mirror 21a, an objective lens 21b, a second semi-transparent mirror 21c, a third semi-transparent mirror 21d, a CCD image sensor 21e, and a plurality of anterior-segment illumination light sources not illustrated. The members (first semi-transparent mirror 21a, objective lens 21b, second semi-transparent mirror 21c, third semi-transparent mirror 21d, and CCD image sensor 21e) except the anterior-segment illumination light sources are disposed on an optical axis O1.

In the anterior-segment observation system 21, the cornea Ec is directly illuminated by the plurality of anterior-segment illumination light sources disposed left and right with respect to the examinee's eye E. The illumination light of the anterior-segment illumination light sources, reflected off the cornea Ec, passes through the first semi-transparent mirror 21a and then converges due to the objective lens 21b. The converging light is transmitted through the second semi-transparent mirror 21c and the third semi-transparent mirror 21d in order and then is projected on the light-receiving face of the CCD image sensor 21e. The computing unit 14a captures the anterior-segment image of the examinee's eye E, on the basis of a projected image detected by the CCD image sensor 21e. Note that the illumination light reflected off the cornea Ec is transmitted through a nozzle 32 from inside to outside, a glass plate 31a, and an optical filter 31b in order in the air blowing unit 30 to be described below, reaching the first semi-transparent mirror 21a.

The XY alignment target projection system 22 includes an alignment light source 22a, a converging lens 22b, an aperture stop 22c, a first pinhole plate 22d, a dichroic mirror 22e that reflects infrared light and transmits visible light, and a projection lens 22f. Here, the dichroic mirror 22e and the projection lens 22f are disposed on an optical axis branching off from the optical axis O1 due to the first semi-transparent mirror 21a. The alignment light source 22a, the converging lens 22b, the aperture stop 22c, and the first pinhole plate 22d are disposed on the optical axis of the reflected light due to the dichroic mirror

In the XY alignment target projection system 22, the alignment light source 22a outputs infrared light. Converging due to the converging lens 22b, the infrared light passes through the aperture stop 22c, leading to the first pinhole plate 22d. A pencil of light having passed through the first pinhole plate 22d is reflected by the dichroic mirror 22e and then is brought into a parallel pencil of light by the projection lens 22f. The parallel pencil of light is reflected by the first semi-transparent mirror 21a, so that the examinee's eye E is irradiated with the parallel pencil of light along the optical axis O1. In the XY alignment target projection system 22, the irradiation light is used as XY alignment target light for alignment in the XV directions.

The fixation target projection system 23 includes a fixation target light source 23a. a second pinhole plate 23b, the dichroic mirror 22e, and the projection lens 22f. The members (fixation target light source 23a, second pinhole plate 23b, dichroic mirror 22e, and projection lens 22f) are disposed on the optical axis branching off from the optical axis O1 due to the first semi-transparent mirror 21a.

In the fixation target projection system 23, the fixation target light source 23a outputs visible light. The visible light passes through the second pinhole plate 23b and the dichroic mirror 22e and then is brought into a parallel pencil of light by the projection lens 22f After reflected by the first semi-transparent mirror 21a, the parallel pencil of light is projected on the fundus of the examinee's eye E along the optical axis O1. In the fixation target projection system 23, the projection light projected on the fundus of the examinee's eye E is used as a fixation target.

The XY alignment detection system 24 includes the third semi-transparent mirror 21d and a first optical sensor 24a. The first optical sensor 24a is a sensor capable of position detection like a position sensitive detector (PSD). The first optical sensor 24a is disposed on an optical axis branching off from the optical axis O1 due to the third semi-transparent mirror 21d.

In the XY alignment detection system 24, the first optical sensor 24a receives the component dividedly reflected by the third semi-transparent mirror 21d, in the XY alignment target light reflected off the surface of the cornea Ec, so that a luminous-point image T1 is formed on the light-receiving face of the first optical sensor 24a. The component transmitted through the third semi-transparent mirror 21d, in the XY alignment target light reflected off the surface of the cornea Ec, forms a luminous-point image 12 on the light-receiving face of the CCD image sensor 21e.

The computing unit 14a computes the deviation in the XV directions of the detection unit 20 to the cornea Ec, on the basis of a detection result of the luminous-point image T1 by the first optical sensor 24a. The computing unit 14a causes the display unit 15 to display a detection result of the luminous-point image 12 by the CCD image sensor 21e together with the anterior-segment image of the examinee's eye E, Therefore, the examiner can visually verify the state of alignment in the XY directions.

The cornea deformation detection system 25 includes the second semi-transparent mirror 21c, a third pinhole plate 25a, and a second optical sensor 25b. The third pinhole plate 25a and the second optical sensor 25b are disposed on an optical axis branching off from the optical axis O1 due to the second semi-transparent mirror 21c.

In the cornea deformation detection system 25, the second optical sensor 25b detects, through the third pinhole plate 25a, a pencil of light reflected by the second semi-transparent mirror 21c. The second optical sensor 25b is a sensor capable of detecting the amount of light like a photodiode. The second optical sensor 25b detects the amount of reflected light from the cornea Ec in applanation due to air puffed from the nozzle 32. The computing unit 14a acquires the amount of deformation of the cornea. Ec from a detection result of the second optical sensor 25b, to acquire the value of intraocular pressure of the examinee's eye E.

As above, the cornea deformation detection system 25 serves as an optical system that detects the reflected light from the cornea Ec in applanation. Thus, the optical axis of the reflected light from the cornea Ec is the principal optical axis of the cornea deformation detection system 25, corresponding to the optical axis O1 of the anterior-segment observation system 21. Thus, hereinafter, the optical axis of the reflected light from the cornea Ec is referred to as the “principal optical axis O1 of the cornea deformation detection system 25”.

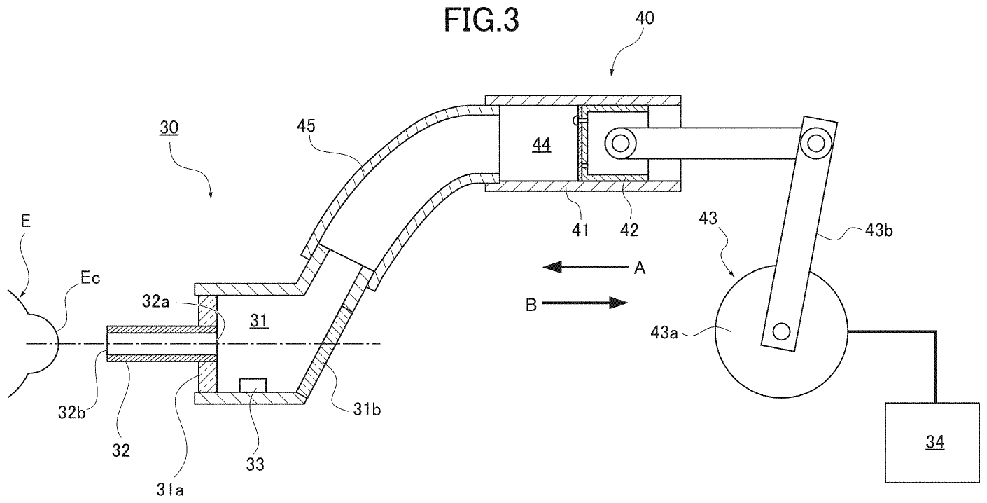

As illustrated in FIG. 3, the air blowing unit 30 includes an air compression chamber 31 that stores air, the nozzle 32, a pressure sensor 33, an air compression unit 40, and the control unit 34. In the air blowing unit 30, the pressure sensor 33 detects the pressure in the air compression chamber 31. The pressure sensor 33 outputs, to the control unit 34, a

signal corresponding to the detected pressure. Then, due to compression of the air in the air compression chamber 31 by the air compression unit 40 under control of the control unit 34, the air blowing unit 30 blows air from the nozzle 32 to the cornea Ec of the examinee's eye E.

The nozzle 32 that is cylindrical in shape with openings at both ends penetrates through the glass plate 31a made of transparent glass provided in the air compression chamber 31. The nozzle 32 has an inlet 32a in communication with the air compression chamber 31 and puffs air from an outlet 32b oriented to the examinee's eye E.

The air compression chamber 31 is provided with the optical filter 31b opposed to the inlet 32a of the nozzle 32. Here, the optical filter 31b made of transparent glass having transparency is located on the principal optical axis O1 of the cornea deformation detection system 25. The central axis of the nozzle 32 (axial direction) is identical to the principal optical axis O1 of the cornea deformation detection system 25. Thus, at the time of detection of the state of deformation of the cornea Ec by the cornea deformation detection system 25, the reflected light from the cornea Ec in applanation transmits through the nozzle 32 and the optical filter 31b.

The air compression unit 40 includes a cylinder 41, a piston 42, and a solenoid actuator (hereinafter, referred to as a “solenoid”) 43. The cylinder 41 that is cylindrical in shape and is hollow houses the piston 42. The solenoid 43 includes a rotary solenoid 43a and a connecting rod 43b. Due to rotary reciprocation of the rotary solenoid 43a, the piston 42 moves in the A or B direction illustrated in FIG. 3 along the axial direction of the cylinder 41. Movement of the piston 42 in the A direction causes compression of the air in a compression chamber 44 inside the cylinder 41. Movement of the piston 42 in the B direction leads to the initial position and additionally causes air to be sucked into the compression chamber 44.

The air compressed in the compression chamber 44 due to movement of the piston 42 flows into the air compression chamber 31 through a tube (or pipe) 45 coupled to the leading end of the cylinder 41 and then puffs out from the nozzle 32 to the examinee's eye E.

The control unit 34 controls the current that flows in the rotary solenoid 43a. (varies current supply duration, voltage, or the like), in accordance with an operation input signal from the operation unit (e.g., display unit 15). Thus, the control unit 34 varies, for example, the direction of movement, speed of movement, or duration of movement of the piston 42, to operation-control, for example, the pressure of a puff of air or the number of puffs of air from the nozzle 32 of the air blowing unit 30. That is, the control unit 34 controls the operation of the air compression unit 40 included in the air blowing unit 30.

Then, the control unit 34 changes the direction of the current that flows in the rotary solenoid 43a, to change the direction of movement of the piston 42. For example, when current flows in the forward direction in the rotary solenoid 43a, the piston 42 moves in the compression direction (in the forward direction, namely, in the A direction of FIG. 3). When current flows in the reverse direction, the piston 42 moves in the opposite direction (in the backward direction, namely, in the B direction of FIG. 3). Thus, for example, the control unit 34 moves the piston 42 in the A direction by sending current in the forward direction in the rotary solenoid 43a, resulting in compression of the air in the compression chamber 44. After that, the control unit 34 moves the piston 42 in the B direction by sending current in the reverse direction in the rotary solenoid 43a, so that the piston 42 can return to the initial position.

The control unit 34 varies the voltage corresponding to the current that flows in the rotary solenoid 43a, to change the speed of movement of the piston 42. That is, the control unit 34 changes the supply current (voltage) to the rotary solenoid 43a, to control deceleration in the speed of return of the piston 42 to the initial position. Thus, the control unit 34 inhibits air from being rapidly sucked into the compression chamber 44 or inhibits interference between the piston 42 and the cylinder 41, so that a loud noise can be inhibited from occurring.

The control unit 34 stops the power supply to the rotary solenoid 43a, to stop the movement of the piston 42. That is, the control unit 34 stops the current supply to the rotary solenoid 43a, so that the operation of puffing air due to the movement of the piston 42 can be stopped.

The control unit 34 stepwise changes (pulse-controls) the supply current to the rotary solenoid 43a, to decelerate the speed of return of the piston 42 to the initial position.

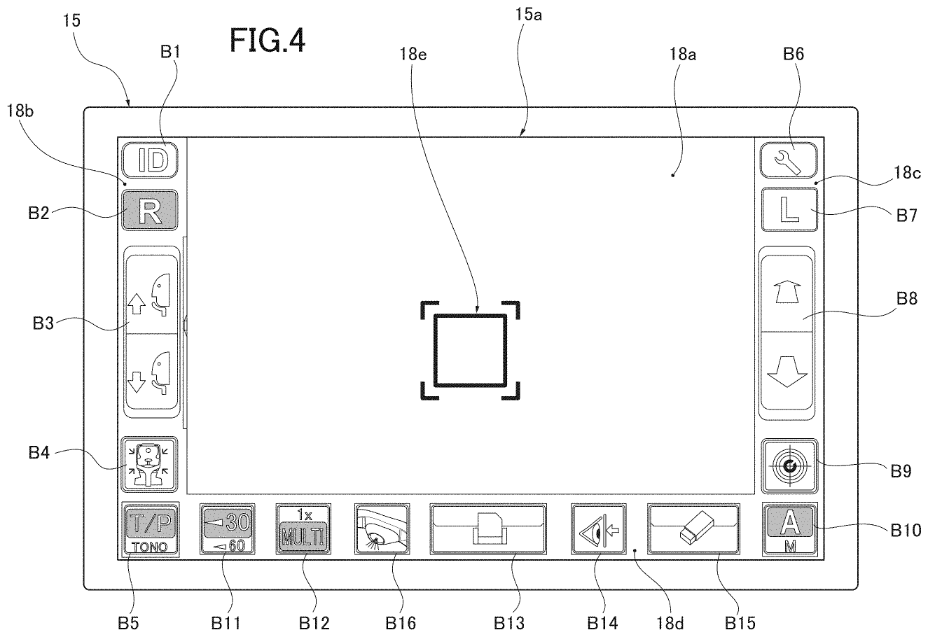

Next, an exemplary screen that the display unit 15 displays on its display will be described with reference to FIG. 4. Note that a screen that the display unit 15 displays is not limited to this example. As illustrated in FIG. 4, a rectangular display 15a is provided with an examinee's eye image/others display area 18a, a first operation-button display area 18b, a second operation-button display area 18c, and a third operation-button display area 18d.

In the examinee's eye image/others display area 18a, a target area mark 18e rectangular in shape is displayed at the center of the screen thereof. In the examinee's eye image/others display area 18a, in addition to the anterior-segment image during observation, displayed appropriately are a measurement result and characters, symbols, marks, or the like related to the examination, moreover, an image, such as a figure. The first operation-button display area 18b and the second operation-button display area 18c are provided, respectively, on the left and right sides of the examinee's eye image/others display area 18a. The third operation-button display area 18d is provided on the lower side thereof.

In the first operation-button display area 18b on the left side, disposed are an ID button B1 for inputting the ID of a patient, an R button B2 for selecting the right eye, a chin-receiver up-and-down movement button B3 for moving the chin receiver 16 upward and downward, an air check button B4 for checking the state of operation of the air compression unit 40, and a measurement-mode setting button B5 for switching the measurement mode of the ophthalmologic apparatus 10. Note that, herein, the measurement mode is switchable between the tono-mode in which the value of intraocular pressure is measured and the pachy-mode in which the thickness of a cornea is measured.

In the second operation-button display area 18c on the right side, disposed are a setup button B6 for displaying a setup screen, an L button B7 for selecting the left eye, a measurement head forward/backward button (Z-direction button) B8 for moving the apparatus main body 13 in the front-and-back direction, a start button B9 for starting measurement in the manual mode, and an automatic/manual switching button B10.

In the third operation-button display area 18d on the lower side, disposed are a pressure switching button B11 for setting the pressure of a puff of air in measurement of the value of intraocular pressure, a number setting button B12 for setting the number of times of measurement in measurement of the value of intraocular pressure, a printout button B13 for printing a measurement result, a safety stopper button B14 for regulating approach of the apparatus main body 13 to the examninee's eye E, and a clearing button B15 for clearing the value of measurement. Furthermore, between the number setting button B12 and the printout button B13, disposed is a preliminary-puffing execution button B16 for manual execution of the preliminary puffing mode to be described below.

Then, the control unit 34 has the measurement mode and the preliminary puffing mode as modes in which the air blowing unit 30 puffs air. Note that, differently from the measurement mode and the preliminary puffing mode, in response to an operation on the air check button B4, the control unit 34 causes the air compression unit 40 to puff air, for verification of whether or not the air compression unit 40 operates properly.

The “measurement mode” causes, at the time of measurement of the value of intraocular pressure of the examinee's eye E by the intraocular-pressure measurement unit 14, the air compression unit 40 to puff air, for applanation of the examinee's eye E. In response to selection of measurement of the value of intraocular pressure by an operation on the measurement-mode setting button B5, the measurement mode is set. The pressure of a puff of air in the measurement mode is set at either 30 or 60 mHg by an operation on the pressure switching button B11.

The “preliminary puffing mode” causes, in non-measurement of the value of intraocular pressure of the examinee's eye E (with the intraocular-pressure measurement unit 14 not performing measurement of the value of intraocular pressure), the air blowing unit 30 to puff air. Note that, in non-measurement of the value of intraocular pressure, for outward discharge of secretions or the like adhering to the inside of the air compression chamber 31 or the inside of the nozzle 32 from the air blowing unit 30, puffing air from the air blowing unit 30 with the air compression unit 40 driving is referred to as “preliminary puffing”.

Then, as the preliminary puffing mode, provided are the “automatic preliminary puffing mode” in which preliminary puffing is automatically executed in response to satisfaction of a previously set preliminary puffing condition and the “manual preliminary puffing mode” in which preliminary puffing is executed in response to an operation on the preliminary-puffing execution button B16 by the examiner. Switching between the automatic preliminary puffing mode and the manual preliminary puffing mode can be made in setting, for example, on the basis of an operation on the automatic/manual switching button B10.

The “preliminary puffing condition” can be set as an arbitrary condition, such as determination of the measurement start of the value of intraocular pressure or determination of the measurement termination of the value of intraocular pressure. The “measurement start of the value of intraocular pressure” corresponds to, for example, determination of execution of measurement of the value of intraocular pressure based on an operation on the measurement-mode setting button B5 by the examiner or a start on alignment of the cornea deformation detection system 25 to the examinee's eye E. The “measurement termination of the value of intraocular pressure” corresponds to, for example, output of measurement data of the value of intraocular pressure or printout of measurement data.

That is, in a case where the preliminary puffing condition corresponds to determination of the measurement start of the value of intraocular pressure, the control unit 34 determines that “the preliminary puffing condition is satisfied”, in response to determination of the timing of the measurement start of the value of intraocular pressure. In a case where the preliminary puffing condition corresponds to determination of the measurement termination of the value of intraocular pressure, the control unit 34 determines that “the preliminary puffing condition is satisfied”, in response to determination of the timing of the measurement termination of the value of intraocular pressure.

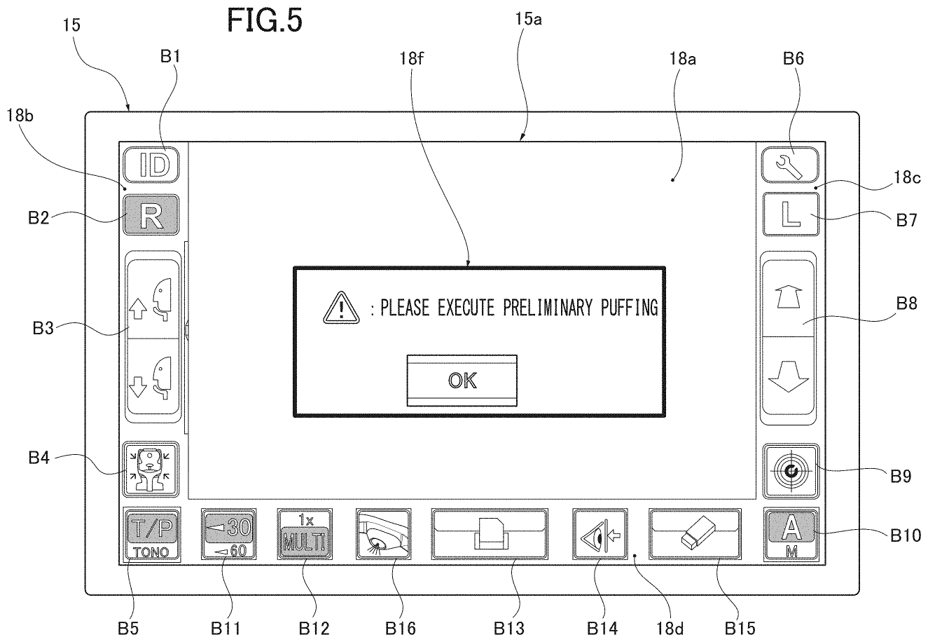

Furthermore, in a case where the preliminary puffing mode based on an operation from the examiner is set (manual preliminary puffing mode), the control unit 34 issues notification that prompts preliminary puffing to be executed, in response to satisfaction of the preliminary puffing condition. Specifically, as illustrated in FIG. 5, a notice window 18f prompting preliminary puffing to be executed is displayed in the examinee's eye image/others display area 18a.

At the time of execution of preliminary puffing, regardless of whether the automatic preliminary puffing mode or the manual preliminary puffing mode is set, the control unit 34 sets the number of puffs of air from the air blowing unit 30, arbitrarily. Note that, for example, the number of puffs of air may be set in accordance with the detail of the preliminary puffing condition or may be set differently between the automatic preliminary puffing mode and the manual preliminary puffing mode. Furthermore, the number of puffs of air may be set in accordance with the number of times of execution of preliminary puffing in a predetermined period.

At the time of execution of preliminary puffing, regardless of whether the automatic preliminary puffing mode or the manual preliminary puffing mode is set, the control unit 34 can set the pressure of a puff of air from the air blowing unit 30 (e.g., 90 mHg) larger than the pressure of a puff of air from the air compression unit 40 in the measurement mode (herein, 30 or 60 mHg). Note that, in a case where the pressure of a puff of air in the measurement mode is set at 30 mHg, for example, the pressure of a puff of air in the preliminary puffing mode may be set at 50 mHg larger than 30 mHg.

At the time of execution of preliminary puffing, regardless of whether the automatic preliminary puffing mode or the manual preliminary puffing mode is set, the control unit 34 performs noise inhibitory control of inhibiting, during execution of preliminary puffing, noise due to the execution of preliminary puffing, Here, the “noise due to the execution of preliminary puffing” corresponds to noise that occurs due to execution of preliminary puffing. Examples thereof include a suction noise that occurs when air is sucked into the cylinder 41, a puff noise that occurs when air is puffed from the nozzle 32, and an interference noise that occurs due to interference between the cylinder 41 and the piston 42. The “noise inhibitory control” is achieved, for example, by the control of deceleration in the speed of return of the piston 42 to the initial position, based on the pulse control of changing stepwise the current that flows in the rotary solenoid 43a, as described above.

Regardless of whether the automatic preliminary puffing mode or the manual preliminary puffing mode is set, the control unit 34 restricts execution of preliminary puffing when the examinee's eye E is present in a predetermined range from the nozzle 32 that puffs air. That is, even when the preliminary puffing condition is satisfied or an operation is made on the preliminary-puffing execution button B16, in a case where it is determined that the examinee's eye E is present near the outlet 32b of the nozzle 32, the control unit 34 does not cause the air compression unit 40 to puff air. Note that whether or not the examinee's eye E is present in the predetermined range from the nozzle 32 is determined, for example, on the basis of whether or not the examinee is in contact with the chin receiver 16 or the forehead support 17, whether or not the XY alignment target light reflected off the surface of the cornea Ec can be received, or other information from a camera or the like. The control unit 34 determines that “the examinee's eye E is present in the predetermined range from the nozzle 32”, in response to detection of the examinee in contact with the chin receiver 16 or the forehead support 17 by a touch sensor or the like, or during reception of the XY alignment target light in the XY alignment detection system 24.

At the time of execution of preliminary puffing, regardless of whether the automatic preliminary puffing mode or the manual preliminary puffing mode is set, the control unit 34 can displace the nozzle 32 that puffs air. Here, the displacement of the nozzle 32 may be achieved by movement of the nozzle 32 from the position at the point in time of execution of displacement in a previously set direction by a predetermined distance or by movement of the nozzle 32 to a previously set position (e.g., the position at a predetermined distance from the chin receiver 16 or the forehead support 17). The displacement of the nozzle 32 may be performed when it is determined that the examinee's eye E is present in the predetermined range from the nozzle 32. In this case, the control unit 34 executes preliminary puffing after displacing the nozzle 32 that puffs air, out of the predetermined range from the examinee's eye E. Note that the control unit 34 controls the drive unit 12 such that the apparatus main body 13 moves in any of the up-and-down direction, the left-and-right direction, and the front-and-back direction, so that the displacement of the nozzle 32 is achieved.

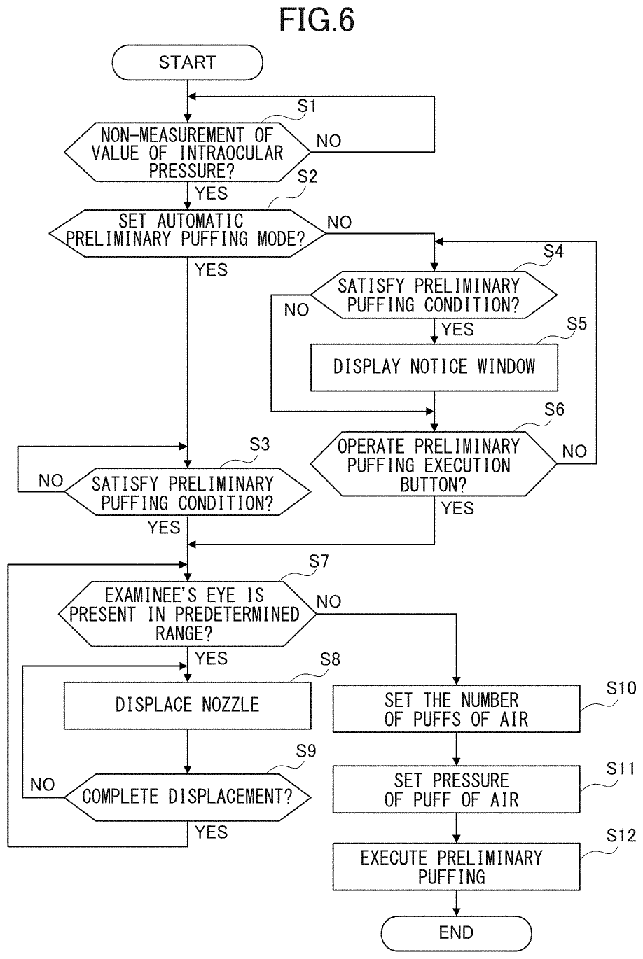

FIG. 6 is a flowchart of preliminary-puffing-mode execution processing that the control unit 34 executes in the first embodiment. Each step in the preliminary-puffing-mode execution processing illustrated in FIG. 6 will be described below

In step S1, the control unit 34 determines whether or not the present is in non-measurement of the value of intraocular pressure (out of measurement of the value of intraocular pressure), In a case where the present is in non-measurement of the value of intraocular pressure (YES), the processing proceeds to step S2. In a case where the present is in measurement of the value of intraocular pressure (NO), step S1 is repeated. Note that, in measurement of the value of intraocular pressure, the control unit 34 is set in the measurement mode.

After the determination in step S1 that the present is in non-measurement of the value of intraocular pressure, in step S2, the control unit 34 determines whether or not the automatic preliminary puffing mode is set. In a case where the automatic preliminary puffing mode is set (YES), the processing proceeds to step S3. In a case where the manual preliminary puffing mode is set (NO), the processing proceeds to step S4.

After the determination in step S2 that the automatic preliminary puffing mode is set, in step S3, the control unit 34 determines whether or not the previously set preliminary puffing condition is satisfied. In a case where the preliminary puffing condition is satisfied (YES), the processing proceeds to step S7. In a case where the preliminary puffing condition is not satisfied (NO), step S3 is repeated.

After the determination in step S2 that the manual preliminary puffing mode is set or after the determination in step S6 that no operation is made on the preliminary-puffing execution button B16, in step S4, the control unit 34 determines whether or not the previously set preliminary puffing condition is satisfied. In a case where the preliminary puffing condition is satisfied (YES), the processing proceeds to step S5. In a case where the preliminary puffing condition is not satisfied (NO), the processing proceeds to step S6.

After the determination in step S4 that the preliminary puffing condition is satisfied, in step S5, the control unit 34 displays the notice window 18f prompting the preliminary puffing to be executed, on the display 15a of the display unit 15. Then, the processing proceeds to step S6.

After the determination in step S4 that the preliminary puffing condition is not satisfied or after the display of the notice window 18f in step S5, in step S6, the control unit 34 determines whether or not an operation is made on the preliminary-puffing execution button B16. In a case where an operation is made on the preliminary-puffing execution button B16 (YES), the processing proceeds to step S7. In a case where no operation is made on the preliminary-puffing execution button B16 (NO), the processing goes back to step S4.

After the determination in step S3 that the preliminary puffing condition is satisfied, after the determination in step S6 that an operation is made on the preliminary-puffing execution button B16, or after the determination in step S9 that the displacement of the nozzle 32 has been completed, in step S7, the control unit 34 determines whether or not the examinee's eye E is present in the predetermined range from the nozzle 32. In a case where the examinee's eye E is present in the predetermined range (YES), the processing proceeds to step S8. In a case where the examinee's eye E is not present in the predetermined range (NO), the processing proceeds to step S10.

After the determination in step S7 that the examinee's eye E is present in the predetermined range, in step S8, the control unit 34 displaces the nozzle 32 from the current position, and then the processing proceeds to step S9. Note that, for achievement of the displacement of the nozzle 32, herein, the control unit 34 drives the drive unit 12 to move the apparatus main body 13 such that the nozzle 32 moves in the previously set direction by the predetermined distance.

After the displacement of the nozzle 32 in step S8, in step S9, the control unit 34 determines whether or not the operation of displacement of the nozzle 32 has been completed. In a case where the displacement has been completed (YES), the processing goes back to step S7. In a case where the displacement has not been completed (NO), step S8 is repeated. Note that whether or not the operation of displacement of the nozzle 32 has been completed is determined on the basis of whether or not the nozzle 32 has moved in the previously set direction by the predetermined distance.

After the determination in step S7 that the examinee's eye E is not present in the predetermined range from the nozzle 32, in step S10, the control unit 34 sets the number of puffs of air in execution of preliminary puffing, and then the processing proceeds to step S11. Here, for example, the number of puffs of air is set in accordance with the detail of the preliminary puffing condition, such as 3 in a case where the preliminary puffing condition corresponds to the “timing of the measurement start of the value of intraocular pressure” and 5 in a case where the preliminary puffing condition corresponds to the “timing of the measurement termination of the value of intraocular pressure. Note that, for example, the number of puffs of air may vary in accordance with the number of times of execution of preliminary puffing in the predetermined period.

After the setting of the number of puffs of air in step S10, in step S11, the control unit 34 sets the pressure of a puff of air in execution of preliminary puffing, and then the processing proceeds to step S12. Here, the pressure of a puff of air is set larger than the pressure of a puff of air, previously set, in the measurement mode. Note that, for example, the pressure of a puff of air may vary in setting in accordance with the detail of the preliminary puffing condition or the number of times of execution of preliminary puffing.

After the setting of the pressure of a puff of air in step S11, in step S12, the control unit 34 supplies current to the rotary solenoid 43a of the air compression unit 40 such that the piston 42 moves, so that preliminary puffing is executed such that air is puffed from the nozzle 32 of the air blowing unit 30. Then, the processing proceeds to the end. At this time, in accordance with the number of puffs of air set in step S10 and the pressure of a puff of air set in step S11, the control unit 34 controls the supply current to the rotary solenoid 43a and simultaneously performs the noise inhibitory control of inhibiting noise due to the execution of preliminary puffing.

Next, effects in the ophthalmologic apparatus 10 according to the first embodiment will be described.

In the ophthalmologic apparatus 10 according to the first embodiment, at the time of measurement of the value of intraocular pressure of the examinee's eye E, the air blowing unit 30 blows air to the cornea Ec of the examinee's eye E. Then, the cornea deformation detection system 25 detects the amount of reflected light from the cornea Ec to which the air is blown, and the computing unit 14a acquires the amount of deformation of the cornea Ec from a detection result, to acquire the value of intraocular pressure of the examinee's eye E.

Here, in some cases, due to air blowing to the cornea Ec, some scattered secretions, such as tears, may flow from the outlet 32b of the nozzle 32 into the air compression chamber 31, and then the secretions may adhere to the inside of the air blowing unit 30, such as the inner face of the air compression chamber 31, the inner face of the optical filter 31b, or the inner circumferential face of the nozzle 32. Thus, the inside of the air blowing unit 30 may be stained. In general, an elongate rod-shaped object, such as a cotton swab, is inserted from the nozzle 32 into the air compression chamber 31, and then the stains are cleaned, with the rod-shaped object, off the inside of the air blowing unit 30 (removal of the stains). However, because the nozzle 32 is considerably narrow and the air compression chamber 31 is small, the cleaning is troublesome,

In order to solve the trouble, in the ophthalmologic apparatus 10 according to the first embodiment, the control unit 34 that controls the operation of the air compression unit 40 of the air blowing unit 30 has the preliminary puffing mode in which air is puffed in non-measurement of the value of intraocular pressure of the examinee's eye E (out of measurement of the value of intraocular pressure) as a mode in which the air blowing unit 30 puffs air.

That is, in the ophthalmologic apparatus 10 according to the first embodiment, first, the control unit 34 determines whether or not the intraocular-pressure measurement unit 14 has been measuring the value of intraocular pressure (step S1). When determining that the intraocular-pressure measurement unit 14 has not been measuring the value of intraocular pressure, the control unit 34 determines whether or not the automatic preliminary puffing mode is set (step S2).

In a case where the control unit 34 determines that the automatic preliminary puffing mode is set, the control unit 34 determines whether or not the previously set preliminary puffing condition is satisfied (step S3). Meanwhile, in a case where the control unit 34 determines that the manual preliminary puffing mode is set, the control unit 34 determines whether or not the previously set preliminary puffing condition is satisfied (step S4). Then, in a case where it is determined that the preliminary puffing condition is satisfied with the manual preliminary puffing mode set, the control unit 34 displays the notice window 18f prompting the preliminary puffing to be executed, on the display 15a of the display unit 15 (step S5).

The control unit 34 determines whether or not an operation is made on the preliminary-puffing execution button B16, with the manual preliminary puffing mode set, regardless of satisfaction of the preliminary puffing condition (step S6).

Then, in a case where the predetermined preliminary puffing condition is satisfied with the automatic preliminary puffing mode set or in a case where an operation is made on the preliminary-puffing execution button B16 with the manual preliminary puffing mode set, for execution of preliminary puffing, first, the control unit 34 determines whether or not the examinee's eye E is present in the predetermined range from the nozzle 32 (step S7). Then, when determining that the examinee's eye E is present in the predetermined range from the nozzle 32, the control unit 34 displaces the nozzle 32 from the current position (steps S8 and S9).

If the examinee's eye E is not present in the predetermined range from the nozzle 32, the control unit 34 sets the number of puffs of air in execution of preliminary puffing (step S10), and then sets the pressure of a puff of air in execution of preliminary puffing (step S11). Then, the control unit 34 controls the operation of the air compression unit 40 on the basis of the previously set number of puffs of air and the previously set pressure of a puff of air, to execute preliminary puffing such that the air blowing unit 30 puffs air. Note that, at this time, the control unit 34 performs the noise inhibitory control of inhibiting noise due to the execution of preliminary puffing.

As above, in the ophthalmologic apparatus 10 according to the first embodiment, execution of preliminary puffing causes the air blowing unit 30 to puff air, so that the strains (secretions) adhering to the inside of the air blowing unit 30, such as the inner face of the air compression chamber 31, the inner face of the optical filter 31b, or the inner circumferential face of the nozzle 32, can be discharged outward from the air blowing unit 30. Thus, strains can be removed from inside the air blowing unit 30, so that inner-strain reduction of the air blowing unit 30 can be achieved.

Furthermore, achievement of inner-strain reduction of the air blowing unit 30 enables a reduction in the number of times of cleaning with a rod-shaped object inserted in the air compression chamber 31 through the nozzle 32, so that reduction of the trouble of cleaning can be achieved.

In the ophthalmologic apparatus 10 according to the first embodiment, in a case where the automatic preliminary puffing mode is set, the preliminary puffing mode is automatically executed in response to satisfaction of the previously set preliminary puffing condition. That is, the control unit 34 automatically executes preliminary puffing in response to satisfaction of the preliminary puffing condition as a trigger.

Thus, even when the examiner makes no operation or verification, preliminary puffing can be executed at an appropriate timing (the timing of satisfaction of the preliminary puffing condition), so that inner-strain reduction of the air blowing unit 30 can be achieved with a reduction in the burden of the examiner.

In the ophthalmologic apparatus 10 according to the first embodiment, in a case where the manual preliminary puffing mode is set, in response to satisfaction of the previously set preliminary puffing condition, the display unit 15 displays the notice window 18f to issue notification that prompts the preliminary puffing to be executed. Thus, even in a case where preliminary puffing is executed by an operation from the examiner (with the manual preliminary puffing mode set), a message that prompts the preliminary puffing to be executed can be given at an appropriate timing (the timing of satisfaction of the preliminary puffing condition), so that the examiner can be prevented from forgetting to execute preliminary puffing.

In the ophthalmologic apparatus 10 according to the first embodiment, the “preliminary puffing condition” is set as either determination of the measurement start of the value of intraocular pressure or determination of the measurement termination of the value of intraocular pressure. Thus, at least either just before the measurement start of the value of intraocular pressure or just after the measurement termination of the value of intraocular pressure, preliminary puffing can be executed.

In a case where preliminary puffing is executed just before the measurement start of the value of intraocular pressure, at the time of measurement of the value of intraocular pressure, because the strains in the air blowing unit 30 are already blown away, it is possible to suppress strain from adhering to the examinee's eye E. Meanwhile, in a case where preliminary puffing is executed just after the measurement termination of the value of intraocular pressure, secretions having entered the air blowing unit 30 can be promptly discharged before being difficult to detach due to drying.

Note that both determination of the measurement start of the value of intraocular pressure and determination of the measurement termination of the value of intraocular pressure may be set as the “preliminary puffing condition”. In this case, preliminary puffing can be executed both before and after measurement of the value of intraocular pressure.

In the ophthalmologic apparatus 10 according to the first embodiment, the control unit 34 sets the number of puffs of air in execution of preliminary puffing, arbitrarily, for example, in accordance with the detail of the preliminary puffing condition. Thus, an appropriate number of puffs of air can be performed in accordance with the state of the air blowing unit 30 at the time of execution of preliminary puffing, so that strains can be effectively discharged.

In the ophthalmologic apparatus 10 according to the first embodiment, the control unit 34 sets the pressure of a puff of air in execution of preliminary puffing larger than the pressure of a puff of air in the measurement mode. Thus, the air blowing unit 30 can puff air intensively, so that strains can be effectively discharged.

In the ophthalmologic apparatus 10 according to the first embodiment, at the time of execution of preliminary puffing, the control unit 34 performs the noise inhibitory control of inhibiting noise due to the execution of preliminary puffing. Thus, noise due to execution of preliminary puffing, such as a suction noise of air, a puff noise of air, or an interference noise between members, can be inhibited, so that quietness can be retained. Thus, a sense of discomfort is less likely to be brought to the examiner or the examinee.

In the ophthalmologic apparatus 10 according to the first embodiment, the control unit 34 restricts execution of preliminary puffing when the examinee's eye E is present in the predetermined range from the nozzle 32 that puffs air. That is, when determining that the examinee's eye E is present near the nozzle 32, the control unit 34 does not execute preliminary puffing. Thus, strains discharged from the air blowing unit 30 due to execution of preliminary puffing can be prevented from adhering to the examinee's eye E. Furthermore, air puffed due to execution of preliminary puffing can be prevented from striking against the examinee's eye E and influencing the examinee's eye E.

Furthermore, in the ophthalmologic apparatus 10 according to the first embodiment, at the time of execution of preliminary puffing, the control unit 34 displaces the nozzle 32 that puffs air, to the predetermined position. That is, the control unit 34 displaces the nozzle 32 with respect to the examinee's eye E and then executes preliminary puffing. Thus, even when the examinee's eye E is present near the ophthalmologic apparatus 10, preliminary puffing can be executed promptly without influence on the examinee's eye E.

The ophthalmologic apparatus according to the first embodiment of the present invention has been described above. The present invention is not limited in specific configuration to the embodiment. Thus, for example, alterations and additions in design may be made without departing from the gist of the invention according to the scope of the claims.

In the first embodiment, only air is puffed in execution of preliminary puffing. However, the present invention is not limited to this. For example, the inside of the air compression chamber 31 or the inside of the nozzle 32 may be irradiated with a beam having disinfecting action or sterilizing action, such as ultraviolet rays. Thus, the air blowing unit 30 can puff air from which bacteria or viruses are eradicated.

Not that, for irradiation of ultraviolet rays or the like, for example, a light source included in the detection unit 20 can be changed with a light source capable of emitting ultraviolet rays or the like, or a light source for ultraviolet rays or the like can be provided in the air compression chamber 31.

In the ophthalmologic apparatus 10 according to the first embodiment, exemplarily, the number of puffs of air and the pressure of a puff of air in the preliminary puffing mode are automatically set before execution of preliminary puffing. However, the present invention is not limited to this. The number of puffs of air or the pressure of a puff of air may be set at an appropriate value by a manual operation by the examiner or may be set at a previously set fixed value. In the automatic preliminary puffing mode, the number of puffs of air or the pressure of a puff of air may be automatically set, for example, in accordance with the detail of the preliminary puffing condition. In the manual preliminary puffing mode, the number of puffs of air or the pressure of a puff of air may be set by a manual operation.

In the ophthalmologic apparatus 10 according to the first embodiment, exemplarily, notification that prompts preliminary puffing to be executed is achieved by display of the notice window 18f. However, the present invention is not limited to this. For example, notification that prompts preliminary puffing to be executed may be achieved by occurrence of a voice or beep or by lighting of a lamp provided on a screen. Notification may be achieved by use of a plurality of means (e.g., display of the notice window 18f and occurrence of a voice).

in the ophthalmologic apparatus 10 according to the first embodiment, exemplarily, no preliminary puffing is executed as restriction of execution of preliminary puffing when the examinee's eye E is present in the predetermined range from the nozzle 32. However, the present invention is not limited in restriction of execution of preliminary puffing to this. For example, preliminary puffing may be executed with the pressure of a puff of air from the air blowing unit 30 smaller with restriction of execution of preliminary puffing than without restriction of execution of preliminary puffing.

Exemplarily, the ophthalmologic apparatus 10 according to the first embodiment is capable of measuring the thickness of a cornea in addition to the value of intraocular pressure. However, the present invention is not limited to this. For example, provided may be an ophthalmologic apparatus capable of measuring only the value of intraocular pressure. Alternatively, provided may be an ophthalmologic apparatus capable of measuring eye characteristics, such as ref (the refractive power of an eye) and kerato (the shape of a cornea), in addition to the value of intraocular pressure.

What is claimed is:

1. An ophthalmologic apparatus that measures a value of intraocular pressure of an examinee's eye, based on a state of deformation of a cornea in applanation due to air blowing, the ophthalmologic apparatus comprising:
an air blowing unit that puffs air to the cornea; and
a control unit that controls an operation of the air blowing unit, wherein
the control unit has a preliminary puffing mode in which preliminary puffing is executed such that the air blowing unit puffs the air in non-measurement of the value of intraocular pressure.

2. The ophthalmologic apparatus according to claim 1, wherein
in response to satisfaction of a previously set preliminary puffing condition, the control unit controls the air blowing unit such that the preliminary puffing is automatically executed.

3. The ophthalmologic apparatus according to claim 1, wherein
in response to satisfaction of a previously set preliminary puffing condition, the control unit issues notification that prompts the preliminary puffing to be executed.

4. The ophthalmologic apparatus according to claim 2, wherein
the control unit sets the preliminary puffing condition as either determination of a measurement start of the value of intraocular pressure or determination of a measurement termination of the value of intraocular pressure.

5. The ophthalmologic apparatus according to claim 1, wherein
the control unit sets a number of puffs of the air in execution of the preliminary puffing, arbitrarily.

6. The ophthalmologic apparatus according to claim 1, wherein
the control unit sets a pressure of a puff of the air in execution of the preliminary puffing larger than a pressure of a puff of the air in measurement of the value of intraocular pressure.

7. The ophthalmologic apparatus according to claim 1, wherein
during execution of the preliminary puffing by the air blowing unit, the control unit performs noise inhibitory control of inhibiting noise due to the execution of the preliminary puffing.

8. The ophthalmologic apparatus according to claim 1, wherein
when the examinee's eye is present in a predetermined range from a nozzle through which the air is puffed, the control unit restricts execution of the preliminary puffing by the air blowing unit.

9. The ophthalmologic apparatus according to claim 1, wherein
when the air blowing unit executes the preliminary puffing, the control unit displaces a nozzle through which the air is puffed, to a predetermined position.
